(19) United States
(12) Patent Application Publication
(10) Pub. No.: US 2015/0065439 A1
(43) Pub. Date: Mar. 5, 2015

(54) PHARMACEUTICAL COMPOSITIONS

(71) Applicant: Vertex Pharmaceuticals Incorporated, Boston, MA (US)

(72) Inventors: Tapan Sanghvi, Watertown, MA (US); Kirk Raymond Dinehart, Holliston, MA (US)

(73) Assignee: Vertex Pharmaceuticals Incorporated, Boston, MA (US)

(21) Appl. No.: 14/191,650

(22) Filed: Feb. 27, 2014

Related U.S. Application Data
(60) Provisional application No. 61/770,471, filed on Feb. 28, 2013.

(51) Int. Cl. A61K 31/7072

(52) U.S. Cl. CPC A61K 31/7072
USPC 514/25

(57) ABSTRACT

Described herein are compositions that can include Compound 1 or Form J of Compound 1, or a pharmaceutically acceptable salt of the aforementioned compounds. Also, described herein is a method of treating a HCV infection in a subject that can include administering to the subject a composition that includes Compound 1 or Form J of Compound 1, or a pharmaceutically acceptable salt of the aforementioned compounds.

This application claims the benefit to U.S. Provisional Application No. 61/770,471, filed on Feb. 28, 2013, which is incorporated herein by reference.

### BACKGROUND

Hepatitis C virus (HCV) is a positive-stranded RNA virus belonging to the Flaviviridae family. HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied.

Combination of pegylated interferon plus ribavirin is the current standard of care for chronic HCV infection. This treatment does not provide sustained viral response (SVR) in a majority of patients infected with the most prevalent genotypes (1a and 1b). Furthermore, significant side effects prevent compliance to the current regimen and may require dose reduction or discontinuation in some patients.

Until recently, the standard of care (SOC) for the treatment of HCV infection comprised 48-week administration of a combination of pegylated interferon-α (subcutaneous weekly injection) and ribavirin (oral, twice daily). Therapy was poorly tolerated and ultimately successful in less than half of the treated patient population. Accordingly, there is a need for the continued development of anti-viral agents and their pharmaceutical compositions for use in treating or preventing Flaviviridae virus infections, such as HCV infections.

### SUMMARY

Some embodiments disclosed herein generally relate to a composition that can include an effective amount of Compound 1 or polymorphic Form J of Compound 1 (hereinafter “Form J”), or a pharmaceutically acceptable salt of the aforementioned compounds. Compound 1 is represented by the following formula structure:

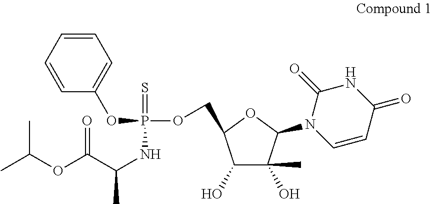 [SMILES: CC(C)OC(=O)[C@H](C)N[P@](=S)(OC[C@H]1O[C@@H](n2ccc(=O)[nH]c2=O)[C@](C)(O)[C@@H]1O)Oc1ccccc1]

Other embodiments disclosed herein generally relate to a method of preparing such compositions described herein (for example, a composition that can include an effective amount of Compound 1 or Form J, or a pharmaceutically acceptable salt of the aforementioned compounds). Still other embodiments disclosed herein generally relate to a method of treating a HCV infection using a composition described herein.

Some embodiments disclosed herein generally relate to a method of inhibiting or reducing the activity of a HCV polymerase in a subject infected with HCV that can include interacting an effective amount of Compound 1 or Form J, or a pharmaceutically acceptable salt of the aforementioned compounds, from a composition described herein with the HCV polymerase.

Other embodiments described herein generally relate to a method of treating a HCV infection in a subject that can include administering to the subject a composition described herein that includes an effective amount of Compound 1 or Form J, or a pharmaceutically acceptable salt of the aforementioned compounds.

Still other embodiments described herein generally relate to a method of inhibiting or reducing the activity of a HCV polymerase or treating a HCV infection in a subject that can include administering to the subject a composition described herein that includes an effective amount of Compound 1 or Form J, or a pharmaceutically acceptable salt of the aforementioned compounds, and one or more additional agents selected from an interferon, ribavirin, a HCV protease inhibitor, a HCV polymerase inhibitor, a NS5A inhibitor, a NS3/4A inhibitor, a viral serine protease inhibitor, a viral helicase inhibitor, an immunomodulating agent, an antioxidant agent, an antibacterial agent, a therapeutic vaccine, a hepatoprotectant agent, an antisense agent, an inhibitor of HCV NS2/3 protease, an inhibitor of internal ribosome entry site (IRES), and an antiviral compound, or a pharmaceutically acceptable salt of any one of the aforementioned compounds.

Some embodiments disclosed herein generally relate to the use of a composition described herein (for example, a composition that includes an effective amount of Compound 1 or Form J, or a pharmaceutically acceptable salt of the aforementioned compounds) in the manufacture of a medicament for treating a HCV infection, or inhibiting or reducing the activity of a HCV polymerase.

### BRIEF DESCRIPTION OF DRAWINGS

FIG. 1 shows an XRPD pattern of Form J.

FIG. 2 shows a DSC thermogram of Form J.

FIG. 3 shows a ¹³C solid state NMR spectrum of Form J.

FIG. 4 shows an XRPD pattern of an amorphous form of Compound 1.

FIG. 5 shows dissolution profile of 50 mg tablets containing Form J in 0.01N HCl.

FIG. 6 shows dissolution profile of 100 mg tablets containing Form J in 50 nM pH 6.8 phosphate buffer with 0.1% SLS.

FIGS. 7A-7J show examples of HCV Protease Inhibitors, Nucleoside and Nucleotide HCV Polymerase Inhibitors, Non-Nucleoside HCV Polymerase Inhibitors, NS5A Inhibitors and other antivirals.

### DETAILED DESCRIPTION

Some embodiments disclosed herein generally relate to a composition that can include an effective amount of Compound 1, or a pharmaceutically acceptable salt thereof; and one or more excipients. Compound 1 is believed to be a NS5B polymerase inhibitor, and described in WO 2012/040127, which is hereby incorporated by reference in its entirety.

Compound 1 and Form J can exist in free form or as a salt. Those salts that are pharmaceutically acceptable can be useful in administering Compound 1 or Form J for medical purposes. Salts that are not pharmaceutically acceptable can be useful for manufacturing, isolating, purifying and/or separating stereoisomeric forms of Compound 1, Form J and/or one or more intermediates thereof.

As used herein, the term “pharmaceutically acceptable salt” refers to a salt of a compound, which are, within the scope of sound medical judgment, suitable for use in humans and lower animals without undue side effects, such as, toxicity, irritation, allergic response and the like, and are commensurate with a reasonable benefit/risk ratio. Various pharmaceutically acceptable salts can be used. For example, those salts disclosed in S. M. Berge et al., J. Pharmaceutical Sciences, 1977, 66, 1-19, which is hereby incorporated by reference. Pharmaceutically acceptable salts of the compounds described herein include those derived from suitable inorganic and organic acids and bases. A salt of a compound described herein (for example, Compound 1) can be prepared in situ during the final isolation and purification of the compound.

Compound 1 can exist in different polymorphic forms. Polymorphism is the ability of a compound to exist as more than one distinct crystalline or “polymorphic” species, wherein each species has a different arrangement of its molecules in the crystal lattice. Each distinct crystalline species is a “polymorph.” Each polymorph has the same chemical formula, however, can be display different physical property(ies) as a result of its different arrangement in the crystal lattice. Polymorphs can be characterized by analytical methods such as X-ray powder diffraction (XRPD) pattern, thermogravimetric analysis (TGA), differential scanning calorimetry (DSC), melting point, and/or other techniques known in the art.

Form J described herein can be in pure form, or in a mixture with other materials. Examples of other materials include, for example, other forms of Compound 1 (such as amorphous forms, other polymorphic forms, solvates and hydrates); other diastereomers of Compound 1; and/or other materials besides Compound 1.

Thus, in some embodiments, a composition can include an effective amount of pure Form J. As used herein, “pure” Form J is over 95% (w/w) (wherein w/w is weight of Form J/weight of Compound 1 (wherein weight of Compound 1 is weight of Form J+weight of all other forms of Compound 1)), for example, over 98% (w/w), over 99% (w/w %), over 99.5% (w/w), or over 99.9% (w/w). In some embodiments, a composition can include an effective amount of Form J in an amount at least 95% (w/w), at least 97% (w/w) or at least 99% (w/w) free of any other diastereomers of Compound 1. In some embodiments, a composition can include an effective amount of Form J in an amount at least 95% (w/w), at least 97% (w/w) or at least 99% (w/w) free of any other polymorphs and amorphous forms of Compound 1.

In some embodiments, a composition can include Form J with one or more other forms of Compound 1. Other forms of Compound 1 include, for example, hydrates, solvates, amorphous forms, other polymorphic forms, or combinations thereof.

In some embodiments, a composition can include an amount of Compound 1 or Form J or a pharmaceutically acceptable salt of the aforementioned compounds in the range of a trace amount (0.1%) up to 100% (w/w) relative to the total weight of the composition. In some embodiments, a composition can include less than about 50% of Compound 1 or Form J or a pharmaceutically acceptable salt of the aforementioned compounds relative to the total weight of the composition (wherein the total weight includes the weight of Compound 1 or Form J or a pharmaceutically acceptable salt of the aforementioned compounds). For example, a composition can include an amount of Compound 1 or Form J or a pharmaceutically acceptable salt of the aforementioned compounds in a range selected from 0.1%-0.5%, 0.1%-1%, 0.1%-2%, 0.1%-5%, 0.1%-10%, 0.1%-20%, 0.1%-30%, 0.1%-40%, and 0.1%-<50% (w/w) relative to the total weight of the composition (wherein the total weight includes the weight of Compound 1 or Form J or a pharmaceutically acceptable salt of the aforementioned compounds). In other embodiments, a composition can include equal to or greater than about 50% of Compound 1 or Form J or a pharmaceutically acceptable salt of the aforementioned compounds relative to the total weight of the composition (wherein the total weight includes the weight of Compound 1 or Form J or a pharmaceutically acceptable salt of the aforementioned compounds). For example, a composition can include at least 50%, 60%, 70%, 80%, 90%, 95%, 97%, 98%, 99%, 99.5% or 99.9% (w/w) of Compound 1 or Form J or a pharmaceutically acceptable salt of the aforementioned compounds relative to the total weight of the composition (wherein the total weight includes the weight of Compound 1 or Form J or a pharmaceutically acceptable salt of the aforementioned compounds). In some embodiments, a composition can include an amount of Compound 1 or Form J or a pharmaceutically acceptable salt of the aforementioned compounds in the range of about 20 wt % to about 70 wt %, about 25 wt % to about 60 wt %, about 30 wt % to about 50 wt %, or about 60 wt % to about 70 wt % of Compound 1 or Form J or a pharmaceutically acceptable salt of the aforementioned compounds relative to the total weight of the composition (wherein the total weight includes the weight of Compound 1 or Form J or a pharmaceutically acceptable salt of the aforementioned compounds).

As used herein, an “excipient” is used herein in its ordinary sense as understood by those skilled in the art, and includes one or more inert substances that are included in a composition to provide, without limitation, bulk, consistency, stability, binding ability, lubrication, disintegrating ability etc., to the composition. Examples of excipients include fillers, binders, disintegrants, wetting agents, lubricants, glidants, humectants and absorbants.

In some embodiments, a composition can include Compound 1 or Form J or a pharmaceutically acceptable salt of the aforementioned compounds and one or more other components selected from one or more fillers, one or more binders, one or more disintegrants, one or more wetting agents and one or more lubricants. In some embodiments, a composition can include an amount of one or more fillers in the range of about 5 wt % to about 70 wt %, %, about 20 wt % to about 35 wt %, about 30 wt % to about 70 wt %, about 35 wt % to about 60 wt %, or about 55 wt % to about 65 wt % of the filler(s) by total weight of the composition (wherein the total weight includes the weight of one or more fillers). In some embodiments, a composition can include an amount of one or more lubricants in the range of about 0.1 wt % to about 10 wt %, about 0.5 wt % to about 7 wt %, or about 1 wt % to about 5 wt % of the lubricant(s) by total weight of the composition (wherein the total weight includes the weight of one or more lubricants). In some embodiments, a composition can include an amount of one or more disintegrants in the range of about 1 wt % to about 15 wt %, about 1 wt % to about 10 wt %, or about 1 wt % to about 5 wt % of the disintegrant(s) by total weight of the composition (wherein the total weight includes the weight of one or more disintegrants). In some embodiments, a composition can include an amount of one or more binders in the range of about 0.25 wt % to about 10 wt %, about 1 wt % to about 10 wt %, or about 1 wt % to about 5 wt % of the binder(s) by total weight of the composition (wherein the total weight includes the weight of one or more binders). In some embodiments, a composition can include an amount of one or more wetting agents in the range of about 0.25 wt % to about 10 wt % or about 1 wt % to about 5 wt % of the wetting agent(s) by total weight of the composition (wherein the total weight includes the weight of one or more wetting agents).

The wetting agents, binders, disintegrants, lubricants and fillers suitable for inclusion can be compatible with the ingredients of the compositions, for example, they do not substantially reduce the chemical stability of the active pharmaceutical ingredient(s).

The term “wetting agent” is used herein in its ordinary sense as understood by those skilled in the art, and includes surfactants, such as non-ionic surfactants and anionic surfactants. Wetting agents can enhance the solubility of the composition. Exemplary surfactants include sodium lauryl sulfate (SLS), polyoxyethylene sorbitan fatty acids (e.g., TWEEN™), sorbitan fatty acid esters (e.g., Spans®), sodium dodecylbenzene sulfonate (SDBS), dioctyl sodium sulfosuccinate (Docusate), dioxycholic acid sodium salt (DOSS), sorbitan monostearate, sorbitan tristearate, sodium N-lauroylsarcosine, sodium oleate, sodium myristate, sodium stearate, sodium palmitate, Gelucire 44/14, ethylenediamine tetraacetic acid (EDTA), Vitamin E d-alpha tocopheryl polyethylene glycol 1000 succinate (TPGS), Lecithin, MW 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, copolymers of polyoxypropylene and polyoxyethylene (e.g., poloxamers also known and commercially available under Pluronics®, such as, Pluronic® L61, Pluronic® F68, Pluronic® F108, and Pluronic® F127), saturated polyglycolized glycerides (Gelucirs®), docusate sodium, polyoxyethylene sorbitan fatty acid esters, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocol derivatives, vitamin E TPGS, tocopheryl esters, lecithin, phospholipids and their derivatives, stearic acid, oleic acid, oleic alcohol, cetyl alcohol, mono and diglycerides, propylene glycol esters of fatty acids, glycerol esters of fatty acids, ethylene glycol palmitostearate, polyoxylglycerides, propylene glycol monocaprylate, propylene glycol monolaurate, polyglyceryl oleate and any combinations thereof. Sodium lauryl sulfate is an anionic surfactant; and copolymers of polyoxypropylene and polyoxyethylene are non-ionic surfactants. Specific examples of copolymers of polyoxypropylene and polyoxyethylene include poloxamers, such as a poloxamer with a polyoxypropylene molecular mass of 1,800 g/mol and a 80% polyoxyethylene content (e.g., poloxamer 188).

The term “binder” is used herein in its ordinary sense as understood by those skilled in the art, and includes agents used while making granules of the active ingredient (for example, Compound 1 or Form J or a pharmaceutically acceptable salt of the aforementioned compounds), wherein a binder holds the active ingredient together with one or more inactive agents. Exemplary binders include polyvinyl pyrrolidones (PVPs), pregelatinized starch, starch, microcrystalline cellulose, modified cellulose (e.g., hydroxyl propyl methyl cellulose (HPMC), hydroxypropyl cellulose (HPC) and hydroxy ethyl cellulose (HEC)), and any combination thereof. PVP's are commonly characterized by the “K-value,” which is a measurement of the polymeric composition's viscosity. PVPs can be commercially purchased (e.g., Tokyo Chemical Industry Co., Ltd.) under the trade name of Povidone® K12, Povidone® K17, Povidone® K25, Povidone® K30, Povidone® K60, and Povidone® K90. Specific examples of PVPs include soluble spray dried PVP. PVPs can have an average molecular weight of 3,000 daltons to 4,000 daltons, such as Povidone® K12 having an average molecular weight of 4,000 daltons. PVP can be used in either a wet or a dry state.

The term “filler” (or “diluent”) is used herein in its ordinary sense as understood by those skilled in the art, and includes microcrystalline celluloses (e.g., Avicel® PH 101), lactoses, sorbitols, celluoses, calcium phosphates, starches, sugars (e.g., mannitol, sucrose, or the like), dextrose, maltodextrin, sorbitol, xylitol, powdered cellulose, silicified microcrystalline cellulose, methylcellulose, ethylcellulose, hydroxyethylcellulose, methylhydroxyethylcellulose, pregelatinized starch, dibasic calcium phosphate, calcium sulfate, calcium carbonate and any combination thereof. Specific examples of fillers include microcrystalline celluloses and lactoses. Specific examples of microcrystalline celluloses include commercially available Avicel® series, such as microcrystalline celluloses having a particle size of 200 mesh over 70% and a particle size of 65 mesh less than 10% (e.g., Avicel® PH 101). A specific example of a lactose is lactose monohydrate.

The term “disintegrant” is used herein in its ordinary sense as understood by those skilled in the art, and can enhance the dispersal of a composition. Examples of disintegrants include croscarmellose sodium, starch (e.g., corn starch, potato starch), sodium starch glycolate, crospovidone, microcrystalline cellulose, sodium alginate, calcium alginate, alginic acid, pregelatinized starch, cellulose and its derivatives, carboxymethylcellulose calcium, carboxymethylcellulose sodium, soy polysaccharide, guar gum, ion exchange resins, an effervescent system based on food acids and an alkaline carbonate component, sodium bicarbonate and any combinations thereof. Specific examples of disintegrants include croscarmellose sodium (e.g., Ac-Di-Sol®) and sodium starch glycolate.

The term “lubricant” is used herein in its ordinary sense as understood by those skilled in the art, and can improve the compression and ejection of a composition, e.g., through a die press. Exemplary lubricants include magnesium stearate, stearic acid (stearin), hydrogenated oils, sodium stearyl fumarate, sodium lauryl sulfate, talc, fatty acid, calcium stearate, sodium stearate, glyceryl monostearate, fatty alcohol, fatty acid ester, glyceryl behenate, mineral oil, vegetable oil, leucine, sodium benzoate and any combination thereof. A specific example of a lubricant is sodium stearyl fumarate.

Those skilled in the art understand that a specific compound described as a wetting agent, binder, filler, disintegrant and lubricant can serve one or more purpose. For example, microcrystalline cellulose can be used as a disintegrant and filler.

In some embodiments, a composition can include an amount of Compound 1 or Form J or a pharmaceutically acceptable salt of the aforementioned compounds in the range of about 20 wt % to about 70 wt % by the total weight of the composition; and an amount of one or more fillers in the range of about 5 wt % to about 70 wt % by the total weight of the composition. In other embodiments, a composition can include an amount of Compound 1 or Form J or a pharmaceutically acceptable salt of the aforementioned compounds in the range of about 20 wt % to about 70 wt % by the total weight of the composition; an amount of one or more fillers in the range of about 5 wt % to about 70 wt % by the total weight of the composition; and an amount of one or more disintegrants in the range of about 1 wt % to about 15 wt % by the total weight of the composition. In still other embodiments, a composition can include an amount of Compound 1 or Form J or a pharmaceutically acceptable salt of the aforementioned compounds in the range of about 20 wt % to about 70 wt % by the total weight of the composition; an amount of one or more fillers in the range of about 5 wt % to about 70 wt % by the total weight of the composition; an amount of one or more disintegrants in the range of about 1 wt % to about 15 wt % by the total weight of the composition; an amount of one or more lubricants in the range of about 0.1 wt % to about 10 wt % by the total weight of the composition; an amount of one or more wetting agents in the range of about 0.25 wt % to about 10 wt % by the total weight of the composition. In yet other embodiments, a composition can include an amount of Compound 1 or Form J or a pharmaceutically acceptable salt of the aforementioned compounds in the range of about 20 wt % to about 70 wt % by the total weight of the composition; an amount of a lubricant in the range of about 0.1 wt % to about 10 wt % by the total weight of the composition; an amount of one or more wetting agents in the range of about 0.25 wt % to about 10 wt % by the total weight of the composition; an amount of a binder in the range of about 0.25 wt % to about 10 wt % by the total weight of the composition; an amount of one or more disintegrants about 1 wt % to about 15 wt % by the total weight of the composition; and an amount of one or more fillers in the range of about 5 wt % to about 70 wt % by the total weight of the composition.

In some embodiments, a composition can include an amount of Compound 1 or Form J or a pharmaceutically acceptable salt of the aforementioned compounds in the range of about 25 wt % to about 60 wt % by the total weight of the composition; an amount of one or more wetting agents in the range of about 0.25 wt % to about 10 wt % by the total weight of the composition; an amount of one or more lubricants in the range of about 1 wt % to about 5 wt % by the total weight of the composition; an amount of one or more disintegrants in the range of about 1 wt % to about 15 wt % by the total weight of the composition; and an amount of one or more fillers in the range of about 25 wt % to about 70 wt % by the total weight of the composition. In other embodiments, a composition can include an amount of Compound 1 or Form J or a pharmaceutically acceptable salt of the aforementioned compounds in the range of about 25 wt % to about 60 wt % by the total weight of the composition; an amount of one or more lubricants in the range of about 1 wt % to about 5 wt % by the total weight of the composition; an amount of one or more wetting agents in the range of about 1 wt % to about 5 wt % by the total weight of the composition; an amount of one or more disintegrants in the range of about 1 wt % to about 5 wt % by the total weight of the composition; and an amount of one or more fillers in the range of about 30 wt % to about 70 wt % by the total weight of the composition. In still other embodiments, a composition can include an amount of Compound 1 or Form J or a pharmaceutically acceptable salt of the aforementioned compounds in the range of about 25 wt % to about 60 wt % by the total weight of the composition; an amount of one or more wetting agents in the range of about 1 wt % to about 5 wt % by the total weight of the composition; an amount of one or more lubricants in the range of about 1 wt % to about 5 wt % by the total weight of the composition; an amount of one or more disintegrants in the range of about 1 wt % to about 5 wt % by the total weight of the composition; and an amount of one or more fillers in the range of about 35 wt % to about 60 wt % by the total weight of the composition. In yet still other embodiments, a composition can include an amount of Compound 1 or Form J or a pharmaceutically acceptable salt of the aforementioned compounds in the range of about 25 wt % to about 60 wt % by the total weight of the composition; an amount of one or more wetting agents in the range of about 1 wt % to about 5 wt % by the total weight of the composition; an amount of one or more lubricants in the range of about 1 wt % to about 5 wt % by the total weight of the composition; an amount of one or more binders in the range of about 1 wt % to about 5 wt % by the total weight of the composition; an amount of one or more disintegrants in the range of about 1 wt % to about 5 wt % by the total weight of the composition; and an amount of one or more fillers in the range of about 35 wt % to about 60 wt % by the total weight of the composition.

In some embodiments, a composition can include an amount of Compound 1 or Form J of about 35 wt % by the total weight of the composition, an amount of lactose monohydrate of about 43 wt % by the total weight of the composition, an amount of Avicel PH-101 (microcrystalline cellulose) of about 14 wt % by the total weight of the composition, an amount of sodium lauryl sulfate of about 1 wt %, by the total weight of the composition, an amount of Ac-Di-Sol (croscarmellose sodium) of about 4 wt % by the total weight of the composition, and an amount of sodium stearyl fumarate of about 3 wt % by the total weight of the composition. In some embodiments, a composition can include an amount of Compound 1 or Form J of about 61 wt % by the total weight of the composition, an amount of microcrystalline cellulose of about 28 wt % by the total weight of the composition, an amount of croscarmellose sodium of about 4 wt % by the total weight of the composition, an amount of sodium lauryl sulfate of about 2 wt % by the total weight of the composition, and an amount of sodium stearyl fumarate of about 3 wt % by the total weight of the composition.

In some embodiments, a composition can further one or more glidants (or “flowing acids”). A glidant enhances the flow properties of a composition by reducing interparticle friction and cohesion. Exemplary glidants include colloidal silicon dioxide, talc, and any combination thereof. A specific example of glidant is amorphous, colloidal silicon dioxide having an average particle size in 0.2-0.3 microns, such as Cab-O-Sil® MSP. The amount of a glidant can vary. For example, the amount of glidant(s) can be in the range of about 0.1 wt % to about 3 wt %, or about 0.1 wt % to about 1 wt % by total weight of the composition (wherein the total weight includes the weight of one or more glidants).

In some embodiments, a composition described herein can further include a coating, such as Opadry II white.

In some embodiments, a composition described herein can be in a solid dosage form, for example, a tablet.

Some embodiments described herein relate to a method of preparing a composition described herein. In some embodiments, a method can include providing a mixture that includes Compound 1 or Form J or a pharmaceutically acceptable salt of the aforementioned compounds and one or more fillers to form a composition. In other embodiments, a method can include providing a mixture that includes Compound 1 or Form J or a pharmaceutically acceptable salt of the aforementioned compounds, a wetting agent, a lubricant, a disintegrant, and a filler to form a composition. Examples, including specific examples, of wetting agents, lubricants, disintegrants, and fillers are each and independently described herein.

In some embodiments, a method can include combining Compound 1 or Form J or a pharmaceutically acceptable salt of the aforementioned compounds and one or more first excipients to form a mixture; and combining the mixture (that includes Compound 1 or Form J or a pharmaceutically acceptable salt of the aforementioned compounds and one or more first excipients) with one or more second excipients. In some embodiments, the first excipients can include one or more of the following: one or more fillers, one or more wetting agents, one or more disintegrants, and one or more lubricants. In some embodiments, the second excipients can include one or more of the following: one or more disintegrants and one or more lubricants.

In other embodiments, a method of preparing a composition described herein can include: i) combining Compound 1 or Form J or a pharmaceutically acceptable salt of the aforementioned compounds; and one or more first excipients that can include one or more fillers, one or more disintegrants and one or more lubricants; and optionally one or more wetting agents to form a mixture, and ii) combining the mixture from i) with one or more second excipients that can include one more disintegrants and one or more lubricants to form a composition. In some embodiments, the one or more first excipients can include an amount of one or more fillers in the range of about 35 wt % to about 60 wt %, an amount of one or more disintegrants in the range of about 0.5 wt % to about 5 wt %, an amount of one or more lubricants in the range of about 1 wt % to about 5 wt %, and optionally an amount of one or more wetting agents in the range of about 1 wt % to about 5 wt % each by the total weight of the composition, and the second excipients can include an amount of one or more lubricants in the range of about 15 wt % to about 50 wt % and an amount of one or more disintegrants in the range of about 0.5 wt % to about 10 wt % each by the total weight of the composition.

In some embodiments, a method of preparing a composition described herein can include: i) providing granules of Compound 1 or Form J or a pharmaceutically acceptable salt of the aforementioned compounds by combining Compound 1 or Form J or a pharmaceutically acceptable salt of the aforementioned compounds; first excipients that can include one or more fillers, one or more disintegrants, one or more lubricants and optionally, one or more wetting agents; and ii) mixing the granules of Compound 1 or Form J or a pharmaceutically acceptable salt of the aforementioned compounds obtained from i) with second excipients that can include one or more disintegrants and one or more lubricants and optionally one or more fillers to form a composition. In some embodiments, the first excipients can include an amount of one or more fillers in the range of about 20 wt % to about 75 wt %, an amount of one or more disintegrants in the range of about 0.5 wt % to about 5 wt %, an amount of a first lubricant in the range of about 1% to about 5% and optionally an amount of a second lubricant in the range of about 1% to about 5% each by the total weight of the composition, and the second excipients can include an amount of one or more third lubricant in the range of about 0.5 wt % to about 10 wt % and an amount of one or more disintegrants in the range of about 0.5 wt % to about 10 wt % each by the total weight of the composition. Examples, including specific examples, of suitable wetting agents, lubricants, disintegrants, and fillers are described herein.

In some embodiments, a method of preparing a composition described herein can include: combining Compound 1 or Form J or a pharmaceutically acceptable salt of the aforementioned compounds with one or more wetting agents to form a first mixture (for example, mixing using a cone mill); combining one or more fillers with one or more disintegrants to form a second mixture (for example, mixing using a cone mill); combining (i) the first mixture of Compound 1 or Form J or a pharmaceutically acceptable salt of the aforementioned compounds and one or more wetting agent(s), (ii) the second mixture one or more fillers and one or more disintegrants, and (iii) a lubricant (for example, combining using a mixer) to form a third mixture, and granulating the third mixture to form granules of Compound 1 or Form J or a pharmaceutically acceptable salt of the aforementioned compounds.

In some embodiments, a method of preparing a composition described herein can include passing one or more disintegrants and one or more lubricants through a sieve; mixing granules of Compound 1 or Form J or a pharmaceutically acceptable salt of the aforementioned compounds with the pre-sieved one or more disintegrants, mixing the granules of Compound 1 or Form J or a pharmaceutically acceptable salt of the aforementioned compounds with the pre-sieved one or more lubricants to form a composition of Compound 1 or Form J or a pharmaceutically acceptable salt of the aforementioned compounds. In some embodiments, a method of preparing a composition described herein can include passing one or more disintegrants and one or more lubricants through a sieve; mixing granules of Compound 1 or Form J or a pharmaceutically acceptable salt of the aforementioned compounds with the pre-sieved one or more disintegrants to form a mixture, and combining one or more pre-sieved lubricants with the mixture of granules of Compound 1 or Form J or a pharmaceutically acceptable salt of the aforementioned compounds and one or more pre-sieved disintegrants to form a composition of Compound 1 or Form J or a pharmaceutically acceptable salt of the aforementioned compounds. The one or more disintegrants and one or more lubricants can be passed through the sieve separately or simultaneously. Additionally, the granules of Compound 1 or Form J or a pharmaceutically acceptable salt of the aforementioned compounds can be combined with one or more pre-sieved disintegrants before, after or simultaneously with one or more pre-sieved lubricants.

In some embodiments, a method of preparing a composition described herein can include compressing granules that include Compound 1 or Form J or a pharmaceutically acceptable salt of the aforementioned compounds through a tablet compression machine to form a tablet that includes Compound 1 or Form J or a pharmaceutically acceptable salt of the aforementioned compounds.

In some embodiments, a tablet that can include Compound 1 or Form J or a pharmaceutically acceptable salt of the aforementioned compounds (for example, the tablets obtained after tablet compression) can be film coated. A suitable coating is Opadry II white.

The compositions described herein may further include one or more pharmaceutically acceptable carriers other than those described previously. As used herein, “pharmaceutically acceptable” means being inert without unduly inhibiting the biological activity of the compounds. The pharmaceutically acceptable carriers should be biocompatible, e.g., non-toxic, non-inflammatory, non-immunogenic or devoid of other undesired reactions or side-effects upon the administration to a subject. Further, standard pharmaceutical formulation techniques can be employed for integrating the aforementioned one or more pharmaceutically acceptable carriers.

Some examples of materials which can serve as pharmaceutically acceptable carriers include, but are not limited to, ion exchangers; alumina; aluminum stearate; lecithin; serum proteins (such as human serum albumin); buffer substances (such as phosphates or glycine); partial glyceride mixtures of saturated vegetable fatty acids; water; salts or electrolytes (such as protamine sulfate, disodium hydrogen phosphate, potassium hydrogen phosphate, sodium chloride, or zinc salts); colloidal silica; magnesium trisilicate; polyacrylates; waxes; polyethylene-polyoxypropylene-block polymers; methylcellulose; hydroxypropyl methylcellulose; wool fat; sugars such as glucose; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; other non-toxic compatible lubricants; coloring agents; releasing agents; sweetening; flavoring agents; perfuming agents; preservatives; sorbents and antioxidants can also be present in the composition, according to the judgment of the formulator.

In some embodiments, at least 50% of a composition described herein dissolves within 30 minutes after complete addition of the composition to a 0.01N HCl solution or a 50 nM pH 6.8 phosphate buffer with 0.1% SLS at 37±0.5° C., optionally with mixing. In some embodiments at least 60%, 70%, 80%, 85%, 90%, 95%, or 99% of a composition described herein dissolves within 30 minutes after complete addition of the composition to a 0.01N HCl solution or a 50 nM pH 6.8 phosphate buffer with 0.1% SLS at 37±0.5° C., optionally with mixing.

Some embodiments described herein relate to a method of inhibiting or reducing the activity of a HCV polymerase in a subject that can include administering to the subject a composition described herein that contains an effective amount of Compound 1 or Form J, or a pharmaceutically acceptable salt the aforementioned compounds.

Other embodiments described herein relate to a method of treating HCV infection in a subject that can include administering to the subject a composition described herein that contains an effective amount of Compound 1 or Form J, or a pharmaceutically acceptable salt the aforementioned compounds.

Still other embodiments described herein relate to a method of inhibiting or reducing the activity of HCV polymerase or treating HCV infection in a subject that can include administering to the subject a composition described herein that contains an effective amount of Compound 1 or Form J, or a pharmaceutically acceptable salt the aforementioned compounds, and one or more additional agents selected from an interferon, ribavirin, a HCV protease inhibitor, a HCV polymerase inhibitor, a NS5A inhibitor, a NS3/4A inhibitor, a viral serine protease inhibitor, a viral helicase inhibitor, an immunomodulating agent, an antioxidant agent, an antibacterial agent, a therapeutic vaccine, a hepatoprotectant agent, an antisense agent, an inhibitor of HCV NS2/3 protease, an inhibitor of internal ribosome entry site (IRES), and an antiviral compound, or a pharmaceutically acceptable salt of any one of the aforementioned compounds.

Yet still other embodiments described herein relate to an use of a composition described herein that contains an effective amount of Compound 1 or Form J, or a pharmaceutically acceptable salt the aforementioned compounds, in the manufacture of a medicament for treating a HCV infection, or inhibiting or reducing the activity of a HCV polymerase.

In some embodiments, substantially all by weight of Compound 1 in a composition described herein can be Form J.

In some embodiments, at least 90% by weight of Compound 1 in a composition described herein can be Form J.

In some embodiments, at least 95% by weight of Compound 1 in a composition described herein can be Form J.

In some embodiments, at least 98% by weight of Compound 1 in a composition described herein can be Form J.

In some embodiments, at least 99% by weight of Compound 1 in a composition described herein can be Form J.

The compositions described herein can be administered to humans and other animals orally, rectally, parenterally, intracisternally, intravaginally, intraperitoneally, topically (as by powders, ointments, or drops), bucally, as an oral or nasal spray, or the like. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. In some embodiments, a composition described herein can be administered orally, intraperitoneally and/or intravenously.

Any orally acceptable dosage form including, but not limited to, capsules, tablets, aqueous suspensions or solutions. In the case of tablets, suitable carriers used include, but are not limited to, lactose and corn starch. Lubricating agents, such as magnesium stearate, and/or wetting agents can be added. When aqueous suspensions are used, the active ingredient can be combined with emulsifying and/or suspending agents. If desired, sweetening, flavoring, coloring agents and/or perfuming agents can be included.

Liquid dosage forms for oral administration include, but are not limited to, pharmaceutically acceptable emulsions, microemulsions, solutions, suspensions, syrups and elixirs. In addition to the active compound, the liquid dosage forms may contain inert excipients, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (such as, cottonseed, groundnut, corn, germ, olive, castor, and sesame oils), glycerol, tetrahydrofurfuryl alcohol, polyethylene glycols and fatty acid esters of sorbitan, and mixtures thereof.

Solid dosage forms for oral administration include capsules (for example, soft and hard-filled gelatin capsules), tablets, pills, powders, and granules. In such solid dosage forms, the active compound can be mixed with at least one inert, pharmaceutically acceptable excipient or carrier such as sodium citrate or dicalcium phosphate and/or a) fillers such as starches, lactose, milk sugar, sucrose, glucose, mannitol, and silicic acid, b) binders such as, for example, carboxymethylcellulose, alginates, gelatin, polyvinyl pyrrolidone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also include a buffering agent.

The solid dosage forms of tablets, dragees, capsules, pills, and granules can be prepared with coatings and shells such as enteric coatings and other coatings known in the pharmaceutical formulating art. They may optionally contain opacifying agents and can also be of a composition that can release the active ingredient(s) only, or preferentially, in a certain part of the intestinal tract, optionally, in a delayed manner. Examples of embedding compositions that can be used include polymeric substances and waxes. The active compound (s) can be in a microencapsulated form with one or more excipients.

Sterile injectable forms may be aqueous or oleaginous suspension. Injectable preparations may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in propylene glycol. Among the acceptable vehicles and solvents that may be employed are water, Ringer's solution, U.S.P. and isotonic sodium chloride solution. In addition, sterile, fixed oils can be employed as a solvent or suspending medium. For this purpose any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Dosage forms for topical or transdermal administration include ointments, pastes, creams, lotions, gels, powders, solutions, sprays, inhalants and patches. The active component can be admixed under sterile conditions with a pharmaceutically acceptable carrier, and any preservatives and/or buffers may be included. Ophthalmic formulation, eardrops, and eye drops can be formulated. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Alternatively, the active compounds and pharmaceutically acceptable compositions thereof may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Surfactants, such as Tweens, Spans and other emulsifying agents or bioavailability enhancers can be included in a solid, liquid and other dosage forms described herein.

The compositions described herein can be formulated in an unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration, for example, from 0.01 mg/kg body weight/day to 100 mg/kg body weight/day.

In some embodiments, a compositions described herein can be in the form of a solid dosage form. In some embodiments, a composition described herein can be in the form of a tablet.

It will be appreciated that the amount of the active compound (for example, Compound 1 or Form J or a pharmaceutically acceptable salt of the aforementioned compounds) required for use in treatment will vary not only with the particular compound selected but also with the route of administration, the nature of the condition for which treatment is required and the age and condition of the subject and will be ultimately at the discretion of the attendant physician or veterinarian. In general, however, a suitable dose will be in the range of from about 0.1 to about 100 mg/kg of body weight per day, for example, in the range of 0.5 to 50 mg/kg/day, or, for example, in the range of 1 to 10 mg/kg/day.

In some embodiments, a composition described herein can be administered in an amount in the range of about 50 mg to about 400 mg of Compound 1 or Form J, or a pharmaceutically acceptable salt the aforementioned compounds, per day.

In some embodiments, a composition described herein can be administered

a) in an amount of about 50 mg Compound 1 or Form J, or a pharmaceutically acceptable salt the aforementioned compounds, per day;

b) in an amount of about 100 mg Compound 1 or Form J, or a pharmaceutically acceptable salt the aforementioned compounds, per day;

c) in an amount of about 150 mg Compound 1 or Form J, or a pharmaceutically acceptable salt the aforementioned compounds, per day;

d) in an amount of about 200 mg Compound 1 or Form J, or a pharmaceutically acceptable salt the aforementioned compounds, per day;

e) in an amount of about 250 mg Compound 1 or Form J, or a pharmaceutically acceptable salt the aforementioned compounds, per day;

f) in an amount of about 300 mg Compound 1 or Form J, or a pharmaceutically acceptable salt the aforementioned compounds, per day;

g) in an amount of about 350 mg Compound 1 or Form J, or a pharmaceutically acceptable salt the aforementioned compounds, per day; or

h) in an amount of about 400 mg Compound 1 or Form J, or a pharmaceutically acceptable salt the aforementioned compounds, per day.

In some embodiments, a composition described herein can be administered in a fasted state (for example, the subject has not eaten food or liquids, except for water, for at least 8 hours). In other embodiments, a composition described herein can be administered in a fed state (for example, with food or within 1 hour of eating food).

The desired dose may conveniently be presented in a single dose or as divided dose administered at appropriate intervals, for example as two, three, four or more doses per day. In some embodiments, a composition described herein can be administered once per day.

Some embodiments described herein relate to a compositions described herein (for example, a composition that includes an effective amount of Compound 1 or Form J, or a pharmaceutically acceptable salt the aforementioned compounds) that can be used for treating or preventing a Flaviviridae viral infection in a host by administering to the host a composition described herein.

The terms “subject,” “host,” or “patient” includes an animal and a human (e.g., male or female, for example, a child, an adolescent, or an adult). Preferably, the “subject,” “host,” or “patient” is a human.

In some embodiments, the Flaviviridae viral infection can be a hepatitis C viral infection (HCV), such as HCV genotype 1, 2, 3, 4, 5, or 6 infection. In some embodiments, the HCV infection can be a HCV genotype 1 infection, such as genotype 1a or genotype 1b.

In some embodiments, a composition described herein can be used for treating or preventing a Flaviviridae viral infection in a host that can include administering to the host (for example, a composition that includes an effective amount of Compound 1 or Form J or a pharmaceutically acceptable salt of the aforementioned compounds), and further administering at least one additional agent chosen from an interferon, ribavirin, a HCV protease inhibitor, a HCV polymerase inhibitor, a NS5A inhibitor, a NS3/4A inhibitor, a viral serine protease inhibitor, a viral helicase inhibitor, an immunomodulating agent, an antioxidant agent, an antibacterial agent, a therapeutic vaccine, a hepatoprotectant agent, an antisense agent, an inhibitor of HCV NS2/3 protease, an inhibitor of internal ribosome entry site (IRES), and an antiviral compound. In some embodiments, a composition described herein (for example, a composition that includes an effective amount of Compound 1 or Form J or a pharmaceutically acceptable salt of the aforementioned compounds) can be used in a method for inhibiting or reducing the activity of a viral polymerase in a host that can include administering the composition to the host.

In some embodiments, a composition described herein can be used in a method for inhibiting or reducing the activity of a viral polymerase in a host that can include administering a composition described herein (for example, a composition that includes an effective amount of Compound 1 or Form J or a pharmaceutically acceptable salt of the aforementioned compounds) and further administering one or more viral polymerase inhibitors. In some embodiments, the viral polymerase can be a Flaviviridae viral polymerase. In some embodiments, the viral polymerase can be a RNA-dependent RNA-polymerase. In some embodiments, the viral polymerase can be a HCV polymerase. In some embodiments, the viral polymerase can be a HCV NS5B polymerase.

As described herein, a composition that includes Compound 1 or Form J, or a pharmaceutically acceptable salt the aforementioned compounds, can be administered in combination with one or more additional agents chosen from an interferon, ribavirin, a HCV protease inhibitor, a HCV polymerase inhibitor, a NS5A inhibitor, a NS3/4A inhibitor, a viral serine protease inhibitor, a viral helicase inhibitor, an immunomodulating agent, an antioxidant agent, an antibacterial agent, a therapeutic vaccine, a hepatoprotectant agent, an antisense agent, an inhibitor of HCV NS2/3 protease, an inhibitor of internal ribosome entry site (IRES), and an antiviral compound.

Examples of viral NS5A inhibitors include BMS-790025 and GSK2336805; examples of non-nucleoside HCV polymerase inhibitors include HCV-796 and VX-222; examples of nucleoside HCV polymerase inhibitors include PSI-7977, R7128/Mericitabine, R1626 and R1479; examples of HCV NS3 protease inhibitors include TMC-435, VX-950/telaprevir and ITMN-191.

In some embodiments, a composition described herein (for example, a composition that includes an effective amount of Compound 1 or Form J, or a pharmaceutically acceptable salt the aforementioned compounds) can be administered simultaneously with one or more additional agents. In other embodiments, a composition described herein can be administered sequentially with one or more additional agents. The composition that includes Compound 1 or Form J, or a pharmaceutically acceptable salt the aforementioned compounds, may be in a separate composition or in a composition that can include one or more additional agent described herein. When at least two additional agents are administered, at least one additional agent can be administered simultaneously (in the same composition or as a separate composition) and at least one agent can be administered sequentially, the active ingredient can be administered simultaneously with all other additional agents (in the same composition or as separate compositions), or the active ingredient can be administered sequentially with all additional agents.

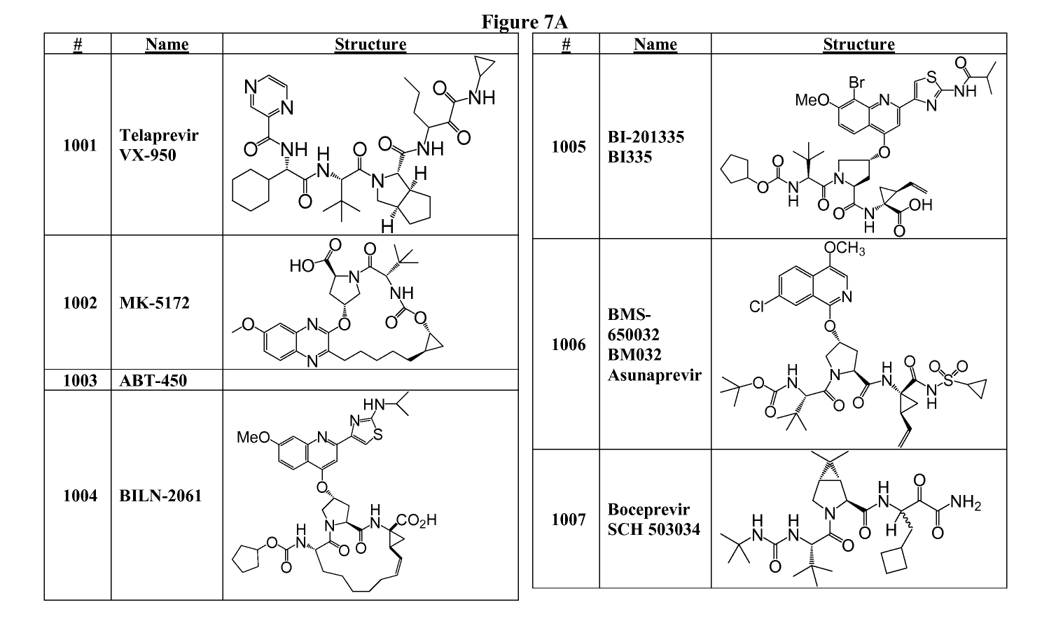

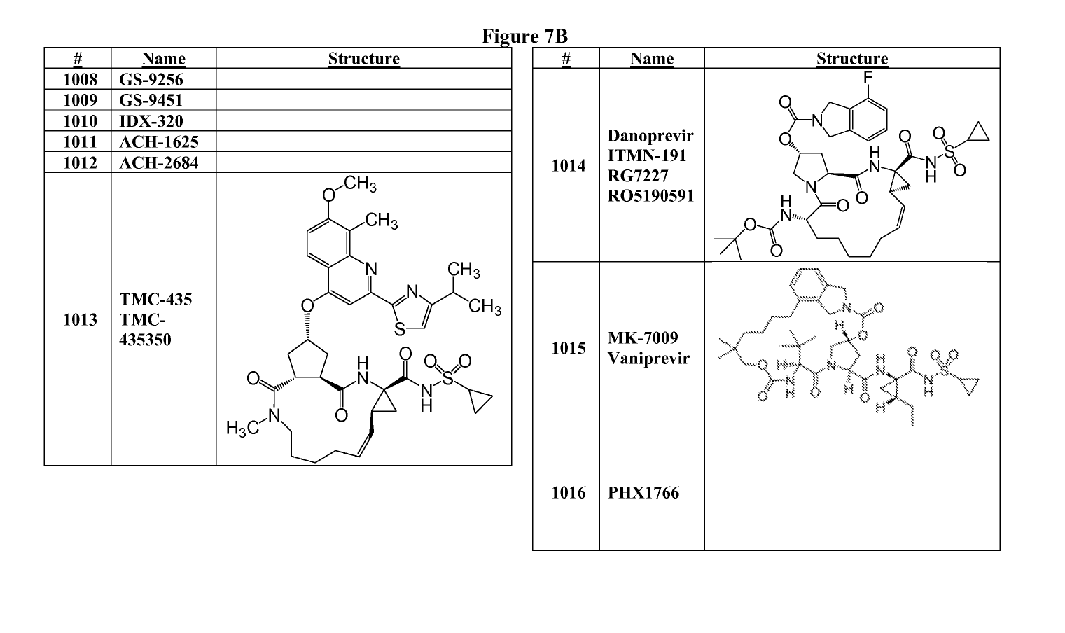

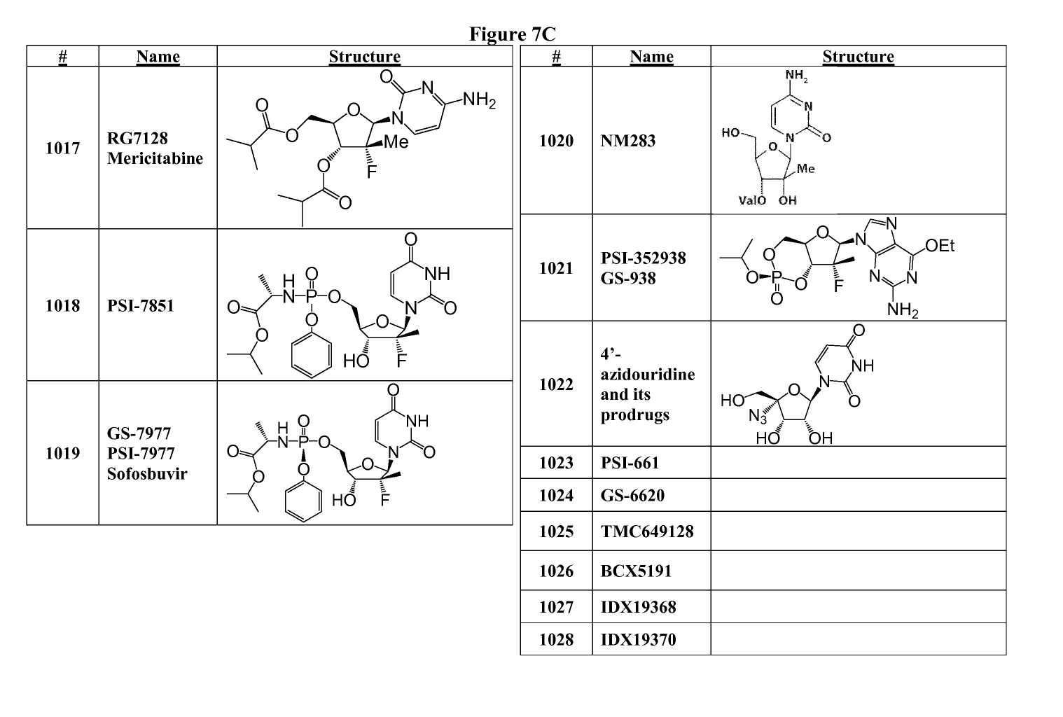

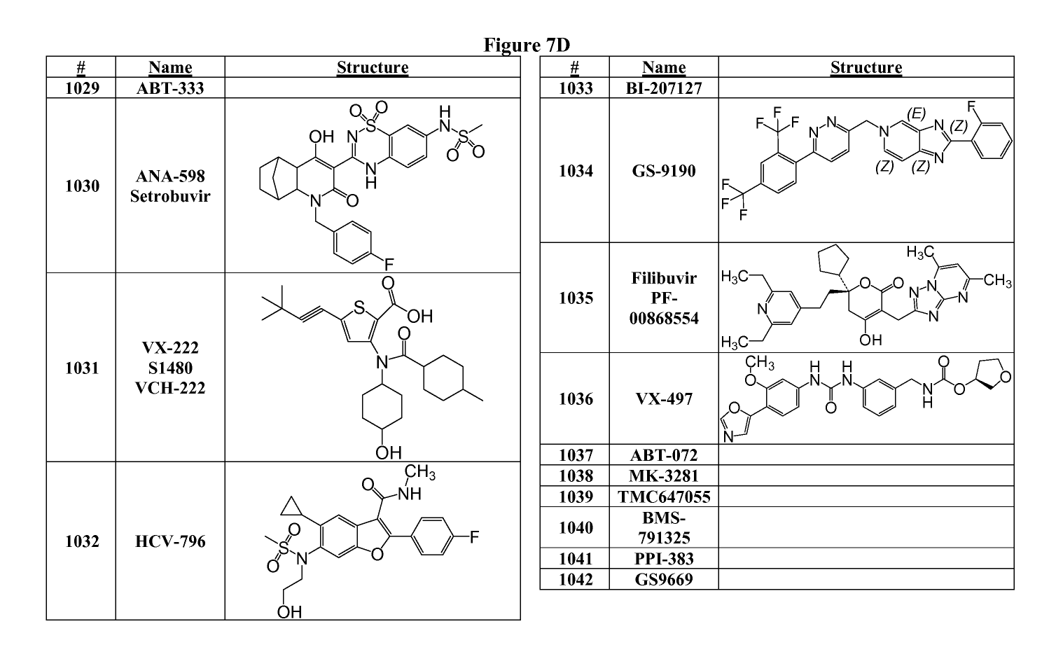

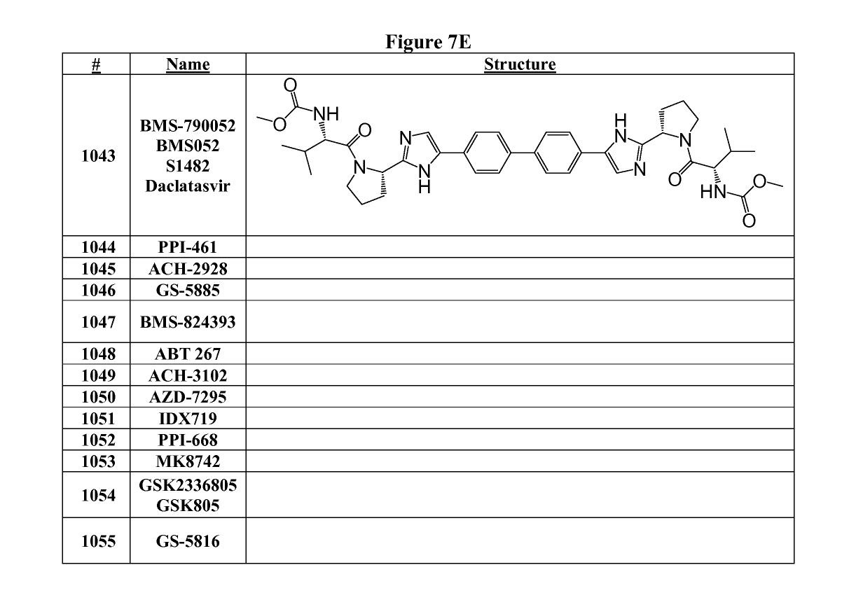

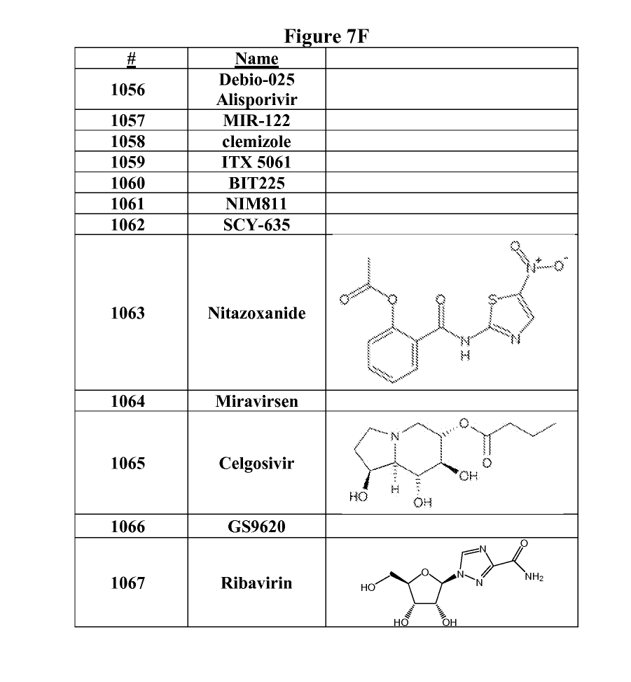

In some embodiments, the additional agent(s) can be selected from Compounds 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066 and 1067 as provided in FIG. 7, or a pharmaceutically acceptable salt of any one of the foregoing.

The term “viral serine protease inhibitor” as used herein means an agent that is effective to inhibit the function of the viral serine protease including HCV serine protease in a mammal Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex), WO 2005007681 (Vertex), WO 2004092162 (Vertex), WO 2004092161 (Vertex), WO 2003035060 (Vertex), WO 03/087092 (Vertex), WO 02/18369 (Vertex), and WO 98/17679 (Vertex).

The term “viral polymerase inhibitors” as used herein means an agent that is effective to inhibit the function of a viral polymerase including an HCV polymerase in a mammal Inhibitors of HCV polymerase include non-nucleosides, for example, those compounds described in: WO 03/010140 (Boehringer Ingelheim), WO 03/026587 (Bristol Myers Squibb); WO 02/100846 A1, WO 02/100851 A2, WO 01/85172 A1 (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Other HCV polymerase inhibitors include nucleoside analogs, for example, those compounds described in: WO 01/90121 A2 (Idenix), WO 02/069903 A2 (Biocryst Pharmaceuticals Inc.), WO 02/057287 A2 (Merck/Isis) and WO 02/057425 A2 (Merck/Isis). Specific examples of nucleoside inhibitors of an HCV polymerase, include PSI-7977/Sofosbuvir (Pharmasset), R1626, R1479 (Roche), R7128 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190 (Gilead), GS-7977 (Gilead), and PF-868,554 (Pfizer).

The term “viral NS5A inhibitor” as used herein means an agent that is effective to inhibit the function of the viral NS5A protease in a mammal. Inhibitors of HCV NS5A include, for example, those compounds described in WO 2010/117635, WO 2010/117977, WO 2010/117704, WO 2010/1200621, WO 2010/096302, WO 2010/017401, WO 2009/102633, WO 2009/102568, WO 2009/102325, WO 2009/102318, WO 2009/020828, WO 2009/020825, WO 2008/144380, WO 2008/021936, WO 2008/021928, WO 2008/021927, WO 2006/133326, WO 2004/014852, WO 2004/014313, WO 2010/096777, WO 2010/065681, WO 2010/065668, WO 2010/065674, WO 2010/062821, WO 2010/099527, WO 2010/096462, WO 2010/091413, WO 2010/094077, WO 2010/111483, WO 2010/120935, WO 2010/126967, WO 2010/132538, and WO 2010/122162. Specific examples of HCV NS5A inhibitors include: EDP-239 (being developed by Enanta); ACH-2928 (being developed by Achillion); PPI-1301 (being developed by Presido Pharmaceuticals); GSK2336805 (being developed by GlaxoSmithKline), PPI-461 (being developed by Presido Pharmaceuticals); AZD-7295 (being developed by AstraZeneca); GS-5885 (being developed by Gilead); BMS-824393 (being developed by Bristol-Myers Squibb); BMS-790052 (Daclatasvir, developed by Bristol-Myers Squibb)

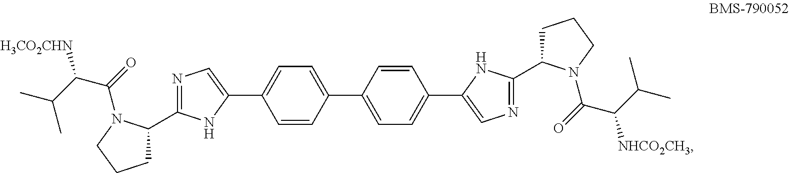

(Gao M. et al. Nature, 465, 96-100 (2010)); and nucleoside or nucleotide polymerase inhibitors, such as PSI-661 (Pharmasset), PSI-938 (Pharmasset), PSI-7977 (Pharmasset), JTK-853 (Japan Tobacco), TMC-647055 (Tibotec Pharmaceuticals), RO-5303253 Hoffmann-La Roche), and IDX-184 (Idenix Pharmaceuticals).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as alpha-interferons, beta-interferons, delta-interferons, omega-interferons, x-interferons, consensus interferons and asialo-interferons), class II interferons (such as gamma-interferons) and pegylated interferons. Examples of immunomodulating agents, include, but are not limited to: thalidomide, IL-2; hematopoietins; IMPDH inhibitors, for example Merimepodib (Vertex Pharmaceuticals Inc.); interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alpha 2a (PEGASYS, Roche), recombinant interferon alpha 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alpha 2b (INTRON A, Schering), pegylated interferon alpha 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs; and synthetic thymosin alpha 1 (ZADAXIN, SciClone Pharmaceuticals Inc.).

The term “class I interferon” as used herein means an interferon selected from interferons that bind to a type 1 receptor. This includes both naturally and synthetically produced class I interferons. Examples of class I interferons include alpha-interferons, beta-interferons, delta-interferons, omega-interferons, tau-interferons, consensus interferons and asialo-interferons. The term “class II interferon” as used herein means an interferon selected from interferons that bind to a type II receptor. Examples of class II interferons include gamma-interferons. Interferons are available in pegylated and non pegylated forms. Pegylated interferons include PEGASYS™ and Peg-intron™.

Antisense agents include, for example, ISIS-14803.

Examples of inhibitors of HCV NS3 protease, include BILN-2061 (Boehringer Ingelheim), SCH-6, SCH-503034/Boceprevir (Schering-Plough), VX-950/telaprevir (Vertex), ITMN-B (InterMune), GS9132 (Gilead), TMC-435350 (Tibotec/Medivir), ITMN-191/Danoprevir (InterMune), and MK-7009/Vaniprevir (Merck).

Inhibitor internal ribosome entry site (IRES) includes ISIS-14803 (ISIS Pharmaceuticals) and those compounds described in WO 2006/019831 (PTC therapeutics).

In some embodiments, the additional agent can be selected from ribavirin, amantadine, merimepodib, Levovirin, Viramidine, and maxamine.

In some embodiments, the additional agent can be selected from interferon alpha, silybum marianum, interleukine-12, amantadine, ribozyme, thymosin, N-acetyl cysteine and cyclosporin.

In some embodiments, the additional agent can be selected from interferon alpha 1A, interferon alpha 1B, interferon alpha 2A, and interferon alpha 2B. In some embodiments, a composition described herein can be administered in combination with an interferon (for example, an interferon selected from interferon alpha 1A, interferon alpha 1B, interferon alpha 2A, and interferon alpha 2B) and ribavirin.

The recommended dose of PEGASYS™ monotherapy for a chronic hepatitis C infection is 180 mg (1.0 mL vial or 0.5 mL prefilled syringe) once weekly for 48 weeks by subcutaneous administration in the abdomen or thigh. The recommended dose of PEG-Intron™ regimen is 1.0 mg/kg/week subcutaneously for one year. The dose should be administered on the same day of the week.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirin tablets are administered at about 1000 mg for subjects weighing less than 75 kg, or at about 1200 mg for subjects weighing more than or equal to 75 kg. Nevertheless, nothing herein limits the methods or combinations to any specific dosage forms or regime. Typically, ribavirin can be dosed according to the dosage regimens described in its commercial product labels. When administered in combination with ribavirin, the recommended dose of PEG-Intron™ is 1.5 micrograms/kg/week.

In some embodiments, Compound 1 or Form J, or a pharmaceutically acceptable salt the aforementioned compounds can be administered with compound 1013. In some embodiments, Compound 1 or Form J, or a pharmaceutically acceptable salt the aforementioned compounds can be administered with compound 1043.

When a composition that includes Compound 1 or Form J, or a pharmaceutically acceptable salt the aforementioned compounds, is used in combination with at least one additional agents, the dose of the composition that includes Compound 1 or Form J, or a pharmaceutically acceptable salt thereof, and/or additional agent(s) may be either the same as or differ from that when the compound is used alone as monotherapy.

As used herein, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

It is understood that, in any compound described, all tautomeric forms are also intended to be included.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are intended to be included. Such compounds are useful, for example, as analytical tools or probes in biological assays. Such compounds, especially deuterium (D) analogs, can also be therapeutically useful.

The compounds described herein are defined herein by their chemical structures and/or chemical names. Where a compound is referred to by both a chemical structure and a chemical name, and the chemical structure and chemical name conflict, the chemical structure is determinative of the compound's identity.

It will be appreciated by those skilled in the art that a compound described herein can exists as stereoisomers (for example, diastereomers or enantiomers), geometrical (cis and trans) and conformational isomers (axial and equatorial). If an absolute stereochemistry is not expressly indicated, all such stereoisomers are intended to be included. Additionally, the compounds provided herein may be enantiomerically pure, enantiomerically enriched, racemic mixture, diastereomerically pure, diastereomerically enriched, or a stereoisomeric mixture.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### General Methods of XRPD, DSC and Solid State NMR

### XRPD (X-ray Powder Diffraction)

Unless otherwise specified, samples were scanned on the Bruker D8 Discover operated at 40 kV, 35 mA. Two frames were registered with an exposure of 120 seconds. Data were integrated over the range of 4.5°-39.0° 2-theta with a step size of 0.02° and merged into one continuous pattern. All XRPD spectra provided herein are measured on a degrees 2-theta scale.

### Differential Scanning Calorimetry (DSC)

The following DSC method was used:

1: Data storage: Off

### 2: Equilibrate at −20.00° C. or 25.00° C.

3: Modulate+/−1.00° C. every 60 seconds

### 4: Isothermal for 5.00 min

5: Data storage: On

### 6: Ramp 2.00-3.00° C./min to 250.00° C.

### Solid State ¹³C Nuclear Magnetic Spectroscopy

Samples were packed into Bruker-Biospin 4 mm ZrO₂ rotors (approximately 65 mg or less each depending on sample availability). The rotors were spun inside a Bruker-Biospin 4 mm HFX probe, which was placed in 400 MHz Bruker-Biospin wide bore magnet. Magic angle spinning (MAS) speed of typically 12.5 kHz was used (10.0 kHz if a suspension was characterized instead of a dry powder). The samples were referenced to adamantine at 29.5 ppm. The proton relaxation time was measured using ¹H MAS T₁ saturation recovery relaxation experiment in order to set up proper recycle delay of the ¹³C cross-polarization (CP) MAS experiment. The CP contact time was set to 2 ms. A CP proton pulse with linear ramp (from 50% to 100%) was employed. The Hartmann-Hahn match was optimized on external reference sample of glycine. SPINAL 64 decoupling was used with the field strength of approximately 90 kHz.

### Example 1

### Preparation of Compound 1 and Form J

### Synthesis of 2′-C-methyluridine 5′-(O-phenyl-N-(S)-1-(isopropoxycarbonyl)ethyl)thiophosphoramidate (Compound 1)

### Method A

Compound 1 can be prepared as described in Example 3 of WO 2012/040127:

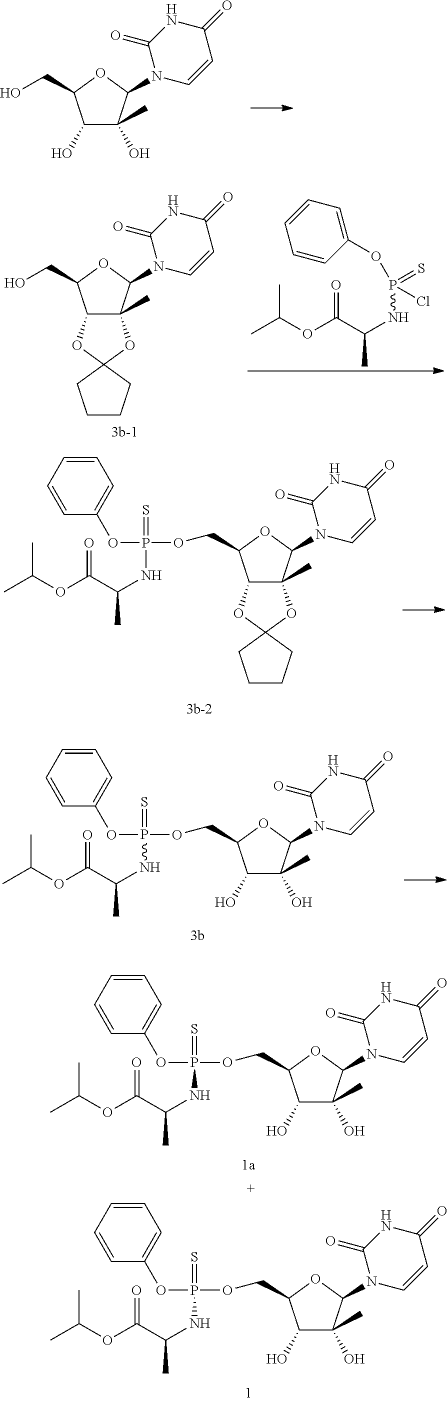

### Method B

50 g of Compound 3b (mixture of two P-diastereomers) was added to 100 mL of DCM and allowed to stir. After brief stirring, almost all of the material was dissolved (<100 mg remained suspended). The mixture was filtered and 300 mL of MTBE added with stirring. About 25 mg of Compound 1 was added as seeds, and the solution was cooled to 3° C. overnight. Precipitation was observed. The cold mixture was filtered, and the solid washed with 25 mL of MTBE. The product was dried on a rotovac at 8 torr and 30° C. This material was recrystallized one additional time using the procedure outlined above with precipitation being observed upon the addition of 100 mg of the product from the first crystallization as seeds. XRPD indicated that the material recovered was amorphous. Additional solids precipitated from the supernatant and were collected by filtration. The solids were then rinsed with 25 mL of MTBE and dried. ³¹P NMR showed that this material was Compound 1 with about 4% of Compound 1a.

### Preparation of Compound 1—Amorphous Form:

350 mg of Compound 1 was weighed and added to 8 mL of a 1:1 DCM/Methanol (HPLC grade) solution in a vial. The contents were allowed to stir until a clear solution was obtained. This solution was spray dried on a Buchi B-290 Mini with a condenser attached. The resulting spray dried solid was further dried in a vacuum oven at room temperature (RT) overnight to remove any residual solvent. The parameters of the Buchi B-290 Mini are listed below:

Nitrogen flow: 10 L/min;

Nitrogen max pressure: 10 psi;

CO₂ max pressure: 15 psi;

Inlet temperature: 95-100° C.;

Outlet temperature: 50° C.;

Aspirator: 100%;

Pump: 30%; and

Nozzle: 1.5

### Preparation of Form J:

To a vial containing 100 mg of Compound 1—amorphous form was added 150 μL of HPLC grade ethanol. The contents of the vial were stirred at an intermediate speed (250 rpm) on a stir plate at RT overnight. The mixture was filtered through a 0.22 μm PVDF filter to provide Form J.

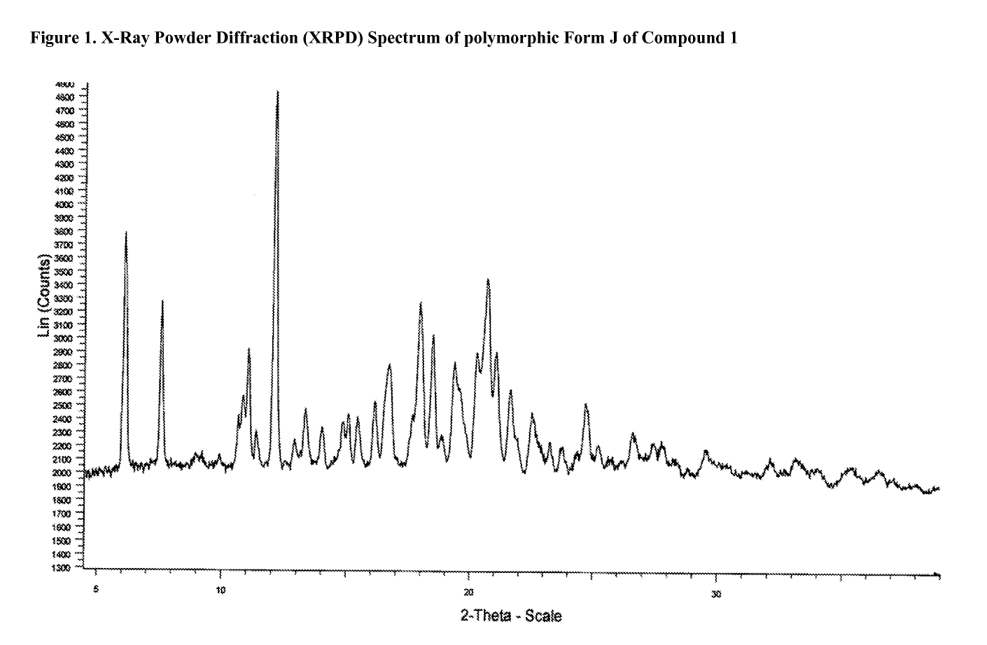

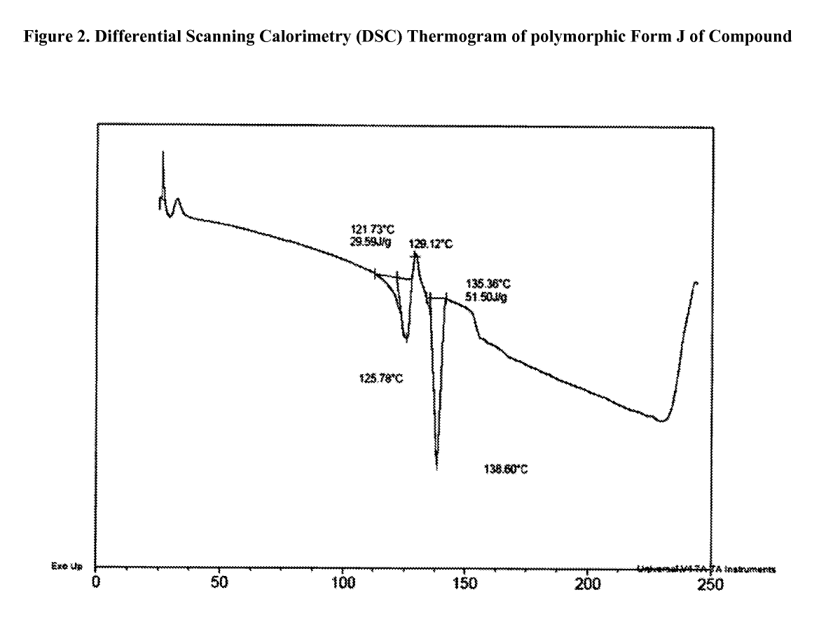

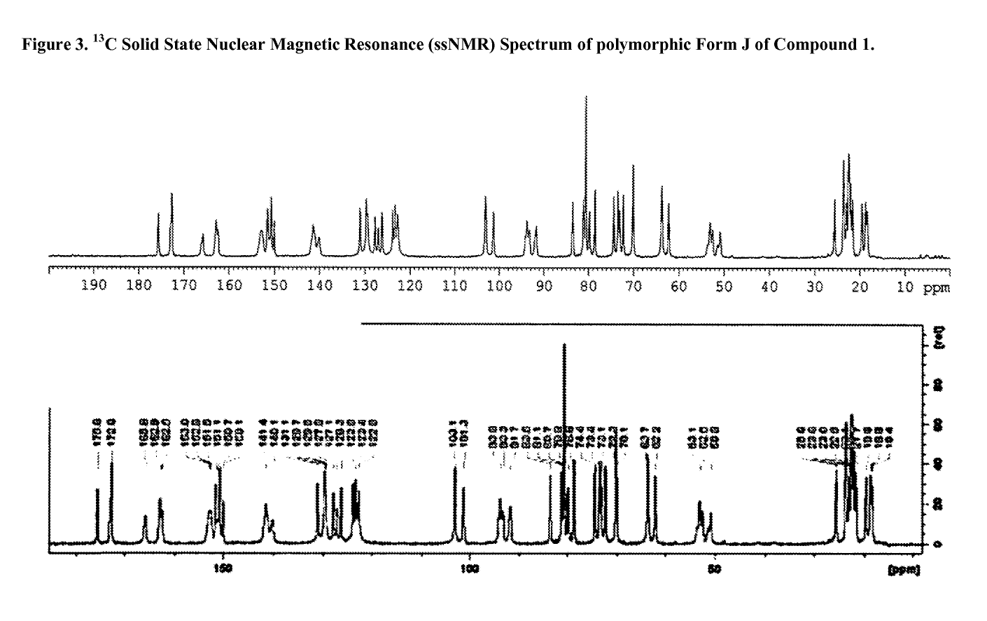

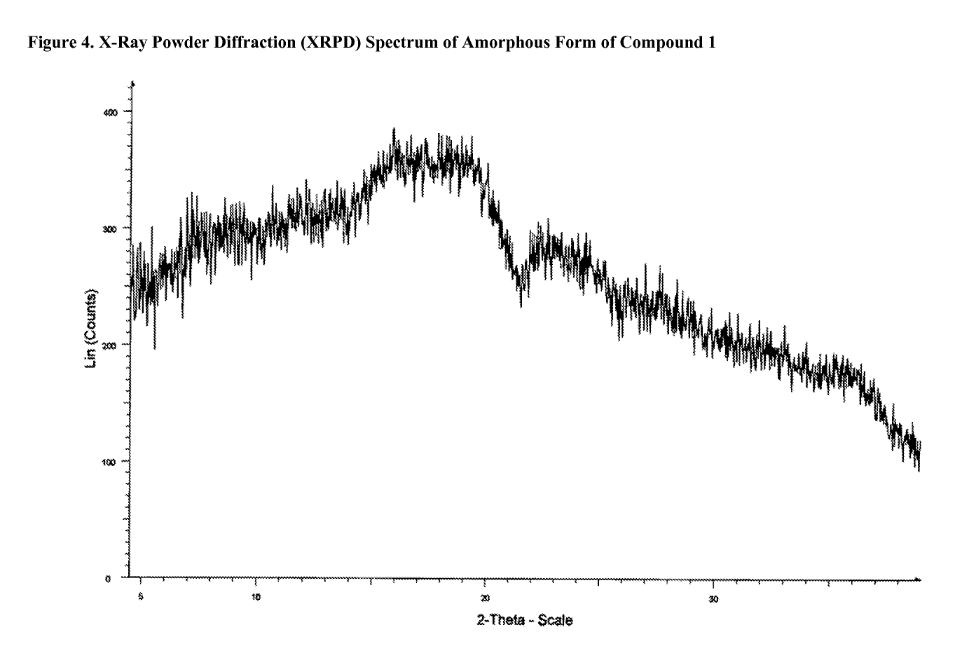

Form J was tested using XRPD, DSC and Solid State ¹³C NMR, which are shown in FIGS. 1-3, respectively.

### Example 2

### Preparation of Tablets of Form J

### Procedure 1

### Dry Granulation and Tablet Composition

The formulation compositions for both the dry granulation and tablet blends of the active tablets are described in Tables 1a and 1b. The overall composition specification of the tablets is described in Table 1c.

TABLE 1a
Form J (50 mg) Dry Granulation Composition

| | Amount (mg) per | |
|---|---|---|
| Component | tablet | % W/W |
| Form J | 50.00 | 35.71 |
| Lactose Monohydrate, #316, NF, PhEur, JP | 61.07 | 43.62 |
| Avicel PH-101 (microcrystalline cellulose), | 20.36 | 14.54 |
| NF, PhEur, JP | | |
| Sodium Lauryl Sulfate | 1.43 | 1.02 |
| NF, PhEur, JP | | |
| Ac-Di-Sol | 4.29 | 3.06 |
| (croscarmellose sodium), NF, PhEur, JP | | |
| Sodium Stearyl Fumarate, NF, PhEur, JP | 2.86 | 2.04 |
| Total | 140.00 | 100.00 |

TABLE 1b
Form J (50 mg) Tablet Composition

| | Amount (mg) per | |
|---|---|---|
| Component | tablet | % W/W |
| Form J Granulation | 140.00 | 98.00 |
| (Milled) | | |
| Ac-Di-Sol | 1.43 | 1.00 |
| (croscarmellose sodium), NF, PhEur, JP | | |
| Sodium Stearyl Fumarate, | 1.43 | 1.00 |
| NF, PhEur, JP | | |
| Total | 142.86 | 100.00 |

TABLE 1c
Form J (50 mg) Tablet Overall Composition*

| | | % in dry | % in core |
|---|---|---|---|
| | | granule | tablet |
| intra | Form J | 35.71 | 35.00 |
| granular | Lactose Monohydrate, #316, NF, PhEur, JP | 43.62 | 42.75 |
| | Avicel PH-101, NF, PhEur, JP | 14.54 | 14.25 |
| | Sodium Lauryl Sulfate, NF, PhEur, JP | 1.02 | 1.00 |
| | Ac-Di-Sol, NF, PhEur, JP | 3.06 | 3.00 |
| | Sodium Stearyl Fumarate, NF, PhEur, JP | 2.04 | 2.00 |
| | total granules: | 100.00 | 98.00 |
| extra | Ac-Di-Sol, NF, PhEur, JP | | 1.00 |
| granular | Sodium Stearyl Fumarate, NF, PhEur, JP | | 1.00 |
| | total core tablet: | | 100.00 |

*The final tablet contained a 3% w/w non-functional coating. The core tablet was 97% w/w of the final tablet.

### Step I. Pre-Granulation Mixing:

Form J was premixed with sodium lauryl sulfate for 25 minutes at 6 rpms. The premix was then passed through a cone mill assembled with a 30 mesh round holed screen and a rounded edge type impeller at an impeller rate of 1400 rpms. Lactose monohydrate, microcrystalline cellulose and intra-granular croscarmellose sodium were passed through the cone mill assembled with a 20 mesh round holed screen and a rounded edge type impeller at an impeller rate of 1400 rpms. The cone milled materials were then mixed for 25 minutes at 6 rpms. Sodium stearyl fumarate was hand sieved through a 60 mesh screen and then charged into the mixer and mixed with the cone milled materials for 15 minutes at 5 rpms.

### Step II. Dry Granulation:

The blend was dry granulated on a Gerteis Minipactor. The blend was passed through the roller compactor, assembled with a combination of smooth faced and knurled faced compaction rolls, at a 2 rpm roll speed with 7KN compaction force and a 2 mm roll gap. Compacted powder was then granulated with a pocketed type milling roll through a 1 mm screen with 100 rpm mill speed.

### Step III. Final Blending:

Extra-granular croscarmellose sodium and sodium stearyl fumarate were hand sieved through 20 and 60 mesh screens, respectively. Extra-granular croscarmellose sodium was blended with the dry granulate for 25 minutes at 6 rpms. Extra-granular sodium stearyl fumarate was then added to the bulk mixture and mixed for 15 minutes at 5 rpms. Samples were pulled for blend uniformity analysis. The blend was sealed in double Low Density Polyethylene bags within a hard secondary container to protect from puncture.

### Step IV. Tablet Compression:

A tablet compression machine (FETTE P2200i) was fully tooled (36 stations) with a 7.0 mm round standard concave tablet punch and die. Tablets were compressed at 70K to 125K tablets per hour. The in-process control testing for tablets included average weight, individual weight, thickness, hardness, friability and disintegration, as shown in Table 1d.

TABLE 1d
Form J (50 mg) Tablet Compression In-process Control Specifications

| Parameter | Minimum | Target | Maximum |
|---|---|---|---|
| Average weight (mg) | 135.8 | 142.9 | 150.0 |
| Individual weight (mg) | 131.5 | 142.9 | 154.3 |
| Thickness (mm) | 3.24 | 3.60 | 3.96 |
| Hardness (SCU) | 5 | 7 | 9 |
| Friability (%) | — | — | 1.0 |
| Disintegration (minutes) | — | — | 30 |

### Step V. Coating:

Tablet cores were coated with Opadry II White 85F, a non-functional opaque white coating of Polyvinyl Alcohol, Polyethylene Glycol, Titanium Dioxide and Talc. The Opadry was mixed with purified water to 20% (w/w) solids, according to manufacturer's instructions. Core tablets, 14 Kg, were charged to a Thomas Compulab coater equipped with a 24″ pan and two 0.9 mm spray nozzles with gun to bed distance of 5″. The tablets were pre-heated to an outlet temperature of 60° C. and then coated to 3% (w/w) coating on the tablets with a 250 CFM inlet air flow, 60° C. inlet temperature, 45° C. outlet temperature, 12 rpm pan speed, 20 mL/min pump rate and 15 psi atomization pressure. In-process samples were pulled to monitor the weight of the coating on the tablets and coating quality. At completion of coating, the tablets were dried for 2 minutes and then cooled down below 30° C. before discharging and packaging.

### Procedure 2

The overall composition specification of the coated tablets is described in Table 2a. The formulation compositions for both the dry granulation and tablet blends of the active tablets are described in Tables 2b-2d.

TABLE 2a
Form J (100 mg) Overall Coated Tablet Composition

| | | | (mg/ |
|---|---|---|---|
| Material | Function | (% w/w) | tablet) |
| Form J | Active | 60.528 | 100.0 |
| Microcrystalline | Filler/Diluent | 27.94 | 46.15 |
| Cellulose | | | |
| Croscarmellose | Intra-Granular Disintegrant | 2.794 | 4.615 |
| Sodium | | | |
| Croscarmellose | Extra-Granular Disintegrant | 0.970 | 1.603 |
| Sodium | | | |
| Sodium Stearyl | Intra-Granular Lubricant | 1.862 | 3.077 |
| Fumarate | | | |
| Sodium Stearyl | Extra-Granular Lubricant | 0.97 | 1.603 |
| Fumarate | | | |
| Sodium Lauryl | Extra-Granular Wetting | 1.940 | 3.205 |
| Sulfate | Agent | | |
| Opadry II White | Non-Functional Coating | 3.000 | 4.956 |
| 85F18422 | | | |

TABLE 2b
Composition for Dry Granulation

| Material | (% w/w) | 16.00 Kg Batch (Kg) |
|---|---|---|
| Form J | 65.00 | 10.40 |
| Microcrystalline Cellulose | 30.00 | 4.80 |
| Intra-Granular Croscarmellose Sodium | 3.00 | 0.48 |
| Intra-Granular Sodium Stearyl Fumarate | 2.00 | 0.32 |

TABLE 2c
Composition for Core Tablet Compression

| Material | (% w/w) | 16.67 Kg Batch (Kg) |
|---|---|---|
| Dry Granulation | 96.00 | 16.00 |
| Sodium Lauryl Sulfate | 2.000 | 0.3333 |
| Extra-Granular Croscarmellose Sodium | 1.000 | 0.1667 |
| Extra-Granular Sodium Stearyl Fumarate | 1.000 | 0.1667 |

TABLE 2d
Composition for Tablet Compression

| Material | (% w/w) | 17.18 Kg Batch (Kg) |
|---|---|---|
| Core Tablets | 97.00 | 16.67 |
| Opadry II White 85F18422 | 3.000 | 0.5155 |

Form J was passed through the cone mill equipped with round impeller and 30 mesh screen at an impeller rate of 1440 rpm into a bin. Intra-granular Microcrystalline cellulose and intra-granular croscarmellose sodium were passed through the cone mill assembled with a 30 mesh round holed screen and a rounded edge type impeller at an impeller rate of 1440 rpms into a bin containing Form J. Sodium stearyl fumarate was hand sieved through a 60 mesh screen and then charged into the bin and mixed with the cone milled materials for 14 minutes at 8 rpms.

The blend was dry granulated on a Gerteis Minipactor. The blend was passed through the roller compactor, assembled with a combination of smooth faced and knurled faced compaction rolls, at a 2 rpm roll speed with 4KN compaction force and a 2 mm roll gap. Compacted powder was then granulated with a pocketed type milling roll through a 1 mm screen with 100 rpm mill speed.

Extra-granular sodium lauryl sulfate and croscarmellose sodium were screened through 20 mesh screen into the blender. Sodium stearyl fumarate was hand sieved through 60 mesh screen into the blender. Extra-granular sodium lauryl sulfate, croscarmellose sodium and sodium stearyl fumarate were blended with the dry granulate for 14 minutes at 8 rpms. Samples were pulled for blend uniformity analysis. The blend was sealed in double Low Density Polyethylene bags within a hard secondary container to protect from puncture.

A tablet compression machine (Courtoy Modul P) was fully tooled (21 stations) with a 7.35 mm round standard concave tablet punch and die. Tablets were compressed at 38K tablets per hour. The in-process control testing for tablets included average weight, individual weight, thickness, hardness, friability and disintegration, as shown in Table 2e.

TABLE 2e
Form J (100 mg) Tablet Compression In-process Control Specifications

| Parameter | Minimum | Target | Maximum |
|---|---|---|---|
| Average weight (mg) | 152.3 | 160.3 | 168.3 |
| Individual weight (mg) | 147.5 | 142.9 | 173.1 |
| Hardness (SCU) | 6 | 9 | 12 |
| Friability (%) | — | — | 1.0 |
| Disintegration (minutes) | — | — | 30 |

Tablet cores were coated with Opadry II White 85F, a non-functional opaque white coating of Polyvinyl Alcohol, Polyethylene Glycol, Titanium Dioxide and Talc. The Opadry was mixed with purified water to 20% (w/w) solids, according to manufacturer's instructions. Core tablets, 5 Kg, were charged to a Thomas Compulab coater equipped with a 19″ pan and one 0.9 mm spray nozzles with gun to bed distance of 5″. The tablets were pre-heated to an exhaust temperature of 45° C. and then coated to 3% (w/w) coating on the tablets with a 225 CFM inlet air flow, 60° C. inlet temperature, 45° C. exhaust temperature, 12 rpm pan speed, 20 mL/min pump rate and 15 psi atomization pressure. In-process samples were pulled to monitor the weight of the coating on the tablets and coating quality. At completion of coating, the tablets were dried for 2 minutes and then cooled down below 30° C. before discharging and packaging.

### Example 3

### Dissolution Testing of Tablets of Form J

Dissolution testing was performed in 0.01N HCl for the 50 mg tablets of Form J prepared in Procedure 1 of Example 2.

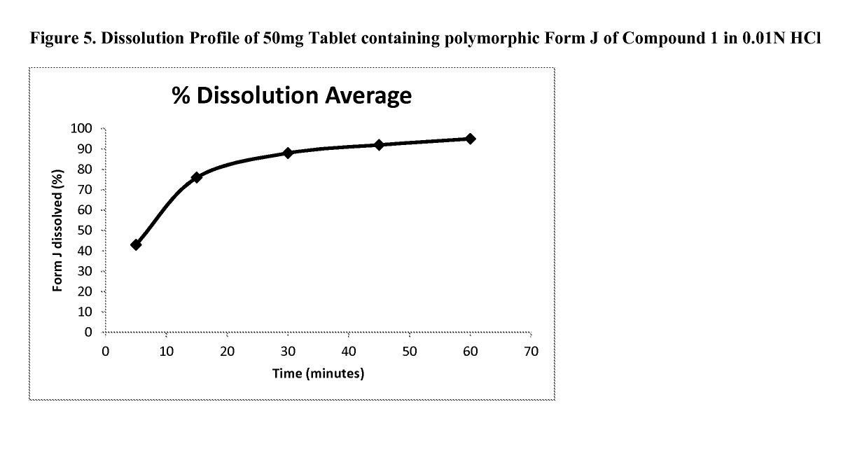

The dissolution profile of the tablets of Form J prepared as described in Procedure 1 is shown in FIG. 5 and is summarized in Table 3a below.

TABLE 3a
Dissolution of 50 mg coated Tablets

| Time | % Dissolution Average |
|---|---|
| 5 | 43 |
| 15 | 76 |
| 30 | 88 |
| 45 | 92 |
| 60 | 95 |

Dissolution testing was performed in 50 nM pH 6.8 phosphate buffer with 0.1% SLS for the 100 mg tablets of Form J prepared in Procedure 2 of Example 2.

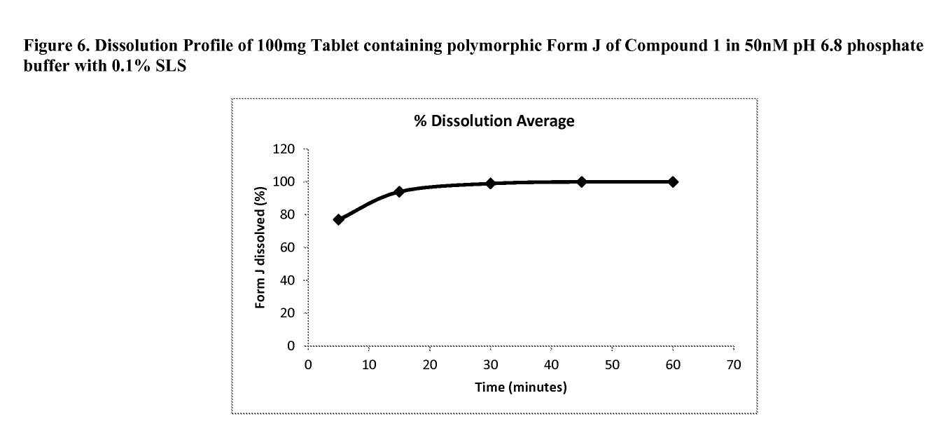

The dissolution profile of the tablets of Form J prepared as described in Procedure 2 is shown in FIG. 6 and is summarized in Table 3b below.

TABLE 3b
Dissolution of 100 mg coated Tablets

| Time | % Dissolution Average |
|---|---|
| 5 | 77 |
| 15 | 94 |
| 30 | 99 |
| 45 | 100 |
| 60 | 100 |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising:
a) Compound 1, or a pharmaceutical acceptable salt thereof, wherein Compound 1 is represented by the following structural formula: 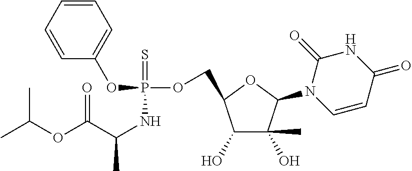 [SMILES: CC(C)OC(=O)[C@H](C)N[P@](=S)(OC[C@H]1O[C@@H](n2ccc(=O)[nH]c2=O)[C@](C)(O)[C@@H]1O)Oc1ccccc1]
and
b) one or more excipients.

2. The composition of claim 2, wherein the one or more excipients comprises one or more selected from the group consisting of one or more fillers, one or more wetting agents, one or more lubricants and one or more disintegrants.

3. The composition of claim 1 or 2, wherein the one or more excipients comprise one or more fillers.

4. The composition of claim 3, wherein the one or more fillers is present in an amount in the range of about 5 wt % to about 70 wt % by the total weight of the composition.

5. The composition of claim 3 or 4, wherein the one or more fillers is selected from the group consisting of mannitol, lactose, sucrose, dextrose, maltodextrin, sorbitol, xylitol, powdered cellulose, microcrystalline cellulose, silicified microcrystalline cellulose, methylcellulose, ethylcellulose, hydroxyethylcellulose, methylhydroxyethylcellulose, starch, pregelatinized starch, dibasic calcium phosphate, calcium sulfate and calcium carbonate.

6. The composition of claim 5, wherein the one or more filler is selected from microcrystalline cellulose and lactose.

7. The composition of any one of claims 1-6, wherein the one or more excipients comprises one or more disintegrants.

8. The composition of claim 7, wherein one or more disintegrants is present in an amount in the range of about 1 wt % to about 15 wt % by the total weight of the composition.

9. The composition of claim 7 or 8, wherein the one or more disintegrants is selected from the group consisting of croscarmellose sodium, sodium alginate, calcium alginate, alginic acid, starch, pregelatinized starch, sodium starch glycolate, crospovidone, cellulose and its derivatives, carboxymethylcellulose calcium, carboxymethylcellulose sodium, soy polysaccharide, guar gum, an ion exchange resin, an effervescent system based on food acids and an alkaline carbonate component, and sodium bicarbonate.

10. The composition of claim 9, wherein the one or more disintegrants is croscarmellose sodium.

11. The composition of claim any one of claims 1-10, wherein the one or more excipients comprises one or more wetting agents.

12. The composition of claim 11, wherein one or more wetting agents is present in an amount in the range of about 0.25 wt % to about 10 wt % by the total weight of the composition.

13. The composition of claim 11 or 12, wherein the one or more wetting agent is selected from the group consisting of sodium lauryl sulfate, docusate sodium, polyoxyethylene sorbitan fatty acid esters, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids, ethylene glycol palmitostearate, polyoxylglycerides, propylene glycol monocaprylate, propylene glycol monolaurate and polyglyceryl oleate.

14. The composition of claim 13, wherein the one or more wetting agent is sodium lauryl sulfate.

15. The composition of any one of claims 1-14, wherein the one or more excipients comprises one or more lubricants.

16. The composition of claim 15, wherein the one or more lubricants is present in an amount in the range of about 0.1 wt % to about 10 wt % by the total weight of the composition.

17. The composition of claim 15 or 16, wherein the one or more lubricants is selected from the group consisting of talc, fatty acid, stearic acid, magnesium stearate, calcium stearate, sodium stearate, glyceryl monostearate, sodium lauryl sulfate, sodium stearyl fumarate, hydrogenated oils, fatty alcohol, fatty acid ester, glyceryl behenate, mineral oil, vegetable oil, leucine, sodium benzoate, and a combination thereof.

18. The composition of claim 17, wherein the one or more lubricants is sodium stearyl fumarate.

19. The composition of any one of claims 1-18, further comprising a coating.

20. The composition of any one of claims 1-19, comprising:
a) an amount of Compound 1 in the range of about 20 wt % to about 70 wt %, or a pharmaceutical acceptable salt thereof, by the total weight of the composition;
b) an amount of one or more wetting agents in the range of about 0.25 wt % to about 10 wt % by the total weight of the composition;
c) an amount of one or more lubricants in the range of about 0.1 wt % to about 10 wt % by the total weight of the composition;
d) an amount of one or more disintegrants in the range of about 1 wt % to about 15 wt % by the total weight of the composition; and
e) an amount of one or more fillers in the range of about 5 wt % to about 70 wt % by the total weight of the composition.

21. The composition of any one of claims 1-20, comprising:
a) an amount of Compound 1 or a pharmaceutical acceptable salt thereof, of about 35 wt % by the total weight of the composition;
b) an amount of lactose monohydrate of about 43 wt % by the total weight of the composition;
c) an amount of Avicel PH-101 (microcrystalline cellulose) of about 14 wt % by the total weight of the composition;
d) an amount of sodium lauryl sulfate of about 1 wt % by the total weight of the composition;
e) an amount of Ac-Di-Sol (croscarmellose sodium) of about 4 wt % by the total weight of the composition; and
e) an amount of sodium stearyl fumarate of about 3 wt % by the total weight of the composition.

22. The composition of any one of claims 1-20, comprising:
a) an amount of Compound 1 or a pharmaceutical acceptable salt thereof, of about 61 wt % by the total weight of the composition;
b) an amount of microcrystalline cellulose of about 28 wt % by the total weight of the composition;
c) an amount of croscarmellose sodium of about 4 wt % by the total weight of the composition;
d) an amount of sodium lauryl sulfate of about 2 wt % by the total weight of the composition; and
e) an amount of sodium stearyl fumarate of about 3 wt % by the total weight of the composition.

23. The composition of any one of claims 1-22, wherein substantially all by weight of Compound 1 is Form J.

24. The composition of claim 23, wherein at least 90% by weight of Compound 1 is Form J.

25. The composition of claim 24, wherein at least 95% by weight of Compound 1 is Form J.

26. The composition of claim 25, wherein at least 98% by weight of Compound 1 is Form J.

27. The composition of any one of claims 1-26, wherein at least 50% of the composition dissolves within 30 minutes after complete addition of the composition to a 0.01N HCl solution at 37±0.5° C.

28. The composition of claim 27, wherein at least 80% of the composition dissolves within 30 minutes after complete addition in a 0.01N HCl solution at 37±0.5° C.

29. A method of treating a HCV infection in a subject, comprising administering to the subject a composition of any one of claims 1-28.

30. A method of inhibiting or reducing the activity of a HCV polymerase in a subject, comprising administering to the subject a composition of any one of claims 1-28.

31. The method of claim 29 or 30, further comprising administering to the subject one or more additional agents selected from the group consisting of an interferon, ribavirin, a HCV protease inhibitor, a HCV polymerase inhibitor, a NS5A inhibitor, a NS3/4A inhibitor, a viral serine protease inhibitor, a viral helicase inhibitor, an immunomodulating agent, an antioxidant agent, an antibacterial agent, a therapeutic vaccine, a hepatoprotectant agent, an antisense agent, an inhibitor of HCV NS2/3 protease, an inhibitor of internal ribosome entry site (IRES), and an antiviral compound, or a pharmaceutically acceptable salt of any one of the aforementioned compounds.

32. The method of claim 31, wherein the one or more additional agents are selected from the group consisting of Compounds 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066 and 1067, or a pharmaceutically acceptable salt of any one of the aforementioned compounds.

33. The method of claim 32, wherein the one or more additional agents is Compound 1043, or a pharmaceutically acceptable salt thereof.

34. The method of any one of claims 29-33, wherein the HCV is selected from the group consisting of genotype 1, 2, 3, 4, 5 and 6.

35. The method of any one of claims 29-34, wherein the composition is administered in an amount in the range of about 50 mg to about 400 mg of Compound 1, or a pharmaceutically acceptable salt thereof.

36. The method of claim 35, wherein the composition is administered in an amount of about 50 mg of Compound 1, or a pharmaceutically acceptable salt thereof; or in an amount of about 100 mg of Compound 1, or a pharmaceutically acceptable salt thereof; or in an amount of about 150 mg of Compound 1, or a pharmaceutically acceptable salt thereof; or in an amount of about 200 mg of Compound 1, or a pharmaceutically acceptable salt thereof; or in an amount of about 250 mg of Compound 1, or a pharmaceutically acceptable salt thereof; or in an amount of about 300 mg of Compound 1, or a pharmaceutically acceptable salt thereof; or in an amount of about 350 mg of Compound 1; or in an amount of about 400 mg of Compound 1, or a pharmaceutically acceptable salt thereof.

37. The method of any one of claims 29-36, wherein the composition is administered once per day.

38. The method of any one of claims 29-34, wherein the composition is administered is in an amount in the range of about 1 mg to about 10 mg of Compound 1 per kilogram of body weight of the subject per day.

39. The method of any one of claims 29-38, wherein the composition is administered in a fasted state.

40. The method of any one of claims 29-38, wherein the composition is administered in in a fed state.
